(19) United States
(12) Patent Application Publication
(10) Pub. No.: US 2026/0183398 A1
(43) Pub. Date: Jul. 2, 2026

(54) RADIOLUMINESCENT NANOPARTICLES FOR RADIATION-TRIGGERED CONTROLLED RELEASE DRUGS

(71) Applicant: Purdue Research Foundation, West Lafayette, IN (US)

(72) Inventors: You-Yeon Won, West Lafayette, IN (US); Jaewon Lee, Richland, WA (US); Rahul Misra, West Lafayette, IN (US); Vincenzo Pizzuti, West Lafayette, IN (US); Kaustabh Sarkar, West Lafayette, IN (US)

(21) Appl. No.: 19/549,970

(22) Filed: Feb. 25, 2026

Related U.S. Application Data
(62) Division of application No. 16/643,607.
(60) Provisional application No. 62/556,289, filed on Sep. 8, 2017.

(51) Int. Cl. A61K 41/00; A61K 9/00; A61K 9/48; A61K 31/337; A61N 5/10; A61P 35/00

(52) U.S. Cl. CPC A61K 41/0042; A61K 9/0019; A61K 9/4816; A61K 31/337; A61N 5/10; A61P 35/00; A61N 2005/1087; A61N 2005/1089; A61N 2005/109; A61N 2005/1098

(57) ABSTRACT

The present disclosure relates to novel radiation-triggered controlled release drug compositions, and methods to make and use the radiation-triggered controlled release drug compositions. The radiation-triggered controlled drug release nanoparticle formulations may be used to achieve maximum bioavailability and minimum adverse effects of the chemo drugs in chemo radio combination therapy treatment of locally advanced solid tumors.

### CROSS-REFERENCE TO RELATED APPLICATION

This application is a divisional of U.S. patent application Ser. No. 16/643,607, filed Mar. 2, 2020, which application is a 35 U.S.C. 371 national phase application of PCT/US2018/49823, filed Sep. 7, 2018, which is related to and claims the benefits of U.S. Provisional application Ser. No. 62/556,289, filed Sep. 8, 2017, the contents of which are incorporated herein entirely.

### TECHNICAL FIELD

The present disclosure relates to novel radiation-triggered controlled release drug compositions, and methods to make and use the radiation-triggered controlled release drug compositions.

### BACKGROUND

This section introduces aspects that may help facilitate a better understanding of the disclosure. Accordingly, these statements are to be read in this light and are not to be understood as admissions about what is or is not prior art.

There has been a steady growth in research on intratumoral chemotherapy during the past couple of decades as an alternative to the conventional systemic delivery approach for patients with unresectable lesions. Intratumoral administration of chemotherapeutic drugs can provide localization of the drugs within the tumor, and can prevent exposure of the non-target organs to such drugs, resulting in reduced toxicity and better efficacy. Intratumoral chemotherapy can be a promising approach not only for the treatment of locally advanced solid tumors but also for malignant gliomas in adjunct therapy.

Polymeric carrier systems are known for their biocompatible nature and ability to sustain the delivery of drugs. The poly(ethylene glycol)-poly(D,L-lactic acid) (PEG-PLA)-based paclitaxel (PTX) formulation, commercially known as Genexol®-PM (Cynviloq™), is an FDA-equivalent-approved example. Intratumoral Pharmacokinetic studies have shown that the polymeric formulation can confine the drug (paclitaxel) within the tumor two times longer than the paclitaxel administered in the form of an organic dispersion.

However, there is still need for a better means to control the drug release rate in order to supply the desired amount of drug to the diseased site on demand and maintain the concentration of the drug inside the tumor within the therapeutically effective range for an extended period of time.

### SUMMARY

The present invention provides novel radiation-triggered controlled release drug compositions, and methods to make and use such compositions.

In one embodiment, the present disclosure provides a radiation-triggered controlled release drug composition comprising:
a) a radio-luminescent particle or particle aggregate capable of emitting UV, visible, IR light, or a combination thereof under radiation;
b) a hydrophobic chemotherapeutic drug; and
c) a biocompatible polymer capsule, wherein the radio-luminescent particle or particle aggregate and the hydrophobic chemotherapeutic drug are co-encapsulated within the biocompatible polymer capsule,
wherein the radio-luminescent particle or particle aggregate emits UV, visible, IR light, or a combination thereof upon receiving a radiation dose, and wherein the radiation directly or indirectly triggers and/or controls the release of the hydrophobic chemotherapeutic drug from the inside of the biocompatible polymer capsule to the outside surrounding tumor tissue.

In another embodiment, the present disclosure provides a method of using a radiation-triggered controlled release drug composition for treating patients with locally advanced primary or metastatic tumors, wherein the method comprises:
a) providing the radiation-triggered controlled release drug composition directly into a tumor, wherein the radiation-triggered controlled release drug composition comprises a radio-luminescent particle or particle aggregate capable of emitting UV, visible, IR light, or a combination thereof under radiation; and a biocompatible polymer capsule, wherein the radio-luminescent particle or particle aggregate and the hydrophobic chemotherapeutic drug are co-encapsulated within the biocompatible polymer capsule; and
b) providing radiation to the tumor that has received the radiation-triggered controlled release drug composition, wherein the radiation triggers the emission of UV, visible, IR light, or a combination thereof from the radio-luminescent particle or particle aggregate, and directly or indirectly triggers the release of the chemotherapeutic drug from the inside of the biocompatible polymer capsule to the outside surrounding tumor tissue.

### BRIEF DESCRIPTION OF THE DRAWINGS

FIG. 1: Schematic illustration of the preparation of PEG-PLA-encapsulated CaWO₄ (CWO) nanoparticles (NPs) loaded with chemotherapeutic drugs, paclitaxel (PTX), and the release of PTX from PEG-PLA/CWO NPs upon exposure to X-Rays.

### DETAILED DESCRIPTION

For the purposes of promoting an understanding of the principles of the present disclosure, reference will now be made to embodiments illustrated in drawings, and specific language will be used to describe the same. It will nevertheless be understood that no limitation of the scope of this disclosure is thereby intended.

In the present disclosure the term “about” can allow for a degree of variability in a value or range, for example, within 10%, within 5%, or within 1% of a stated value or of a stated limit of a range.

In the present disclosure the term “substantially” can allow for a degree of variability in a value or range, for example, within 90%, within 95%, or within 99% of a stated value or of a stated limit of a range.

In the present disclosure the term “radiation” refers to ionizing-radiation or non-ionizing radiation. Ionizing radiation is radiation that carries enough energy to liberate electrons from atoms or molecules, thereby ionizing them. Ionizing radiation may include but is not limited to X-rays, γ rays, electrons, protons, neutrons, ions, or any combination thereof. Non-ionizing radiation refers to any type of electromagnetic radiation that does not carry enough energy per quantum (photon energy) to ionize atoms or molecules—that is, to completely remove an electron from an atom or molecule. Non-ionizing radiation may include but is not limited to ultraviolet (UV), visible, or infrared (IR) light, or any combination thereof. Non-ionizing radiation may be generated by a laser or lamp-type source, and may be delivered directly or by using a fiber optic to the intended delivery site.

Polymeric formulations release encapsulated drugs in a sustained manner. However, there is still need for a better means to control the drug release rate in order to supply the desired amount of drug to the diseased site on demand and maintain the concentration of the drug inside the tumor within the therapeutically effective range for an extended period of time.

The present disclosure provides novel radiation-triggered controlled release drug compositions, and methods to make and use the radiation-triggered controlled release drug compositions.

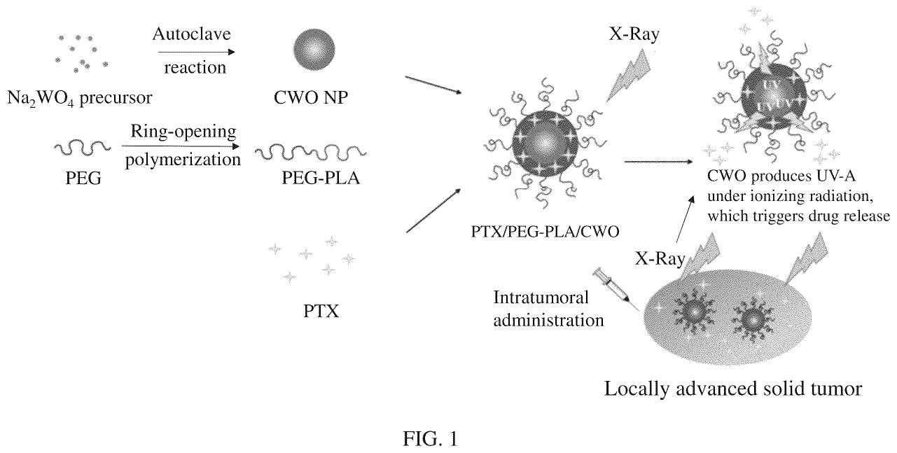

FIG. 1 explains the concept of the novel radiation-triggered controlled release drug composition. Specifically, the FIGURE provides an illustration of the preparation of PEG-PLA-encapsulated CaWO₄ (CWO) nanoparticles (NPs) loaded with chemotherapeutic drugs, paclitaxel (PTX), and the release of PTX from PEG-PLA/CWO NPs upon exposure to X-Rays. CWO NPs are coated with poly(ethylene glycol)-poly(lactic acid) (PEG-PLA) block copolymers. PEG chains are hydrophilic and stay in the aqueous phase. The CWO NP core is coated with hydrophobic PLA chains. PTX is encapsulated within the hydrophobic PLA layer, Under X-ray irradiation, UV-A is generated by CWO NPs, and the X-ray/UV-A causes the release of PTX from the PLA layer into the aqueous surrounding. Intratumorally administered PEG-PLA/CWO/PTX NPs release PTX in tumor during radiation treatments. The PTX release rate is controlled by radiation dose. This concept may be applied to any other combination of choices for radio-luminescent nanoparticles (CaWO₄, ZnO, semiconductor quantum dots, etc.), polyester-based block polymers/light-responsive amphiphiles (PEG-PLA, PEG-PLGA, PEG-PCL, etc.), and hydrophobic chemo drugs (paclitaxel, doxorubicin, cisplatin, etc.).

More specifically, in one embodiment, the present disclosure provides a radiation-triggered controlled release drug composition comprising:
a) a radio-luminescent particle or particle aggregate capable of emitting UV, visible, IR light, or a combination thereof under radiation;
b) a hydrophobic chemotherapeutic drug; and
c) a biocompatible polymer capsule, wherein the radio-luminescent particle or particle aggregate and the hydrophobic chemotherapeutic drug are co-encapsulated within the biocompatible polymer capsule,
wherein the radio-luminescent particle or particle aggregate emits UV, visible, IR light, or a combination thereof upon receiving a radiation dose, and wherein the radiation directly or indirectly triggers and/or controls the release of the hydrophobic chemotherapeutic drug from the inside of the biocompatible polymer capsule to the outside surrounding tumor tissue.

In one embodiment, the present disclosure provides a method of using a radiation-triggered controlled release drug composition for treating patients with locally advanced primary or metastatic tumors, wherein the method comprises:
a) providing the radiation-triggered controlled release drug composition directly into a tumor, wherein the radiation-triggered controlled release drug composition comprises a radio-luminescent particle or particle aggregate capable of emitting UV, visible, IR light, or a combination thereof under radiation, and a biocompatible polymer capsule, wherein the radio-luminescent particle or particle aggregate and the hydrophobic chemotherapeutic drug are co-encapsulated within the biocompatible polymer capsule; and
b) providing radiation to the tumor that has received the radiation-triggered controlled release drug composition, wherein the radiation triggers the emission of UV, visible, IR light, or a combination thereof from the radio-luminescent particle or particle aggregate, and directly or indirectly triggers the release of the chemotherapeutic drug from the inside of the biocompatible polymer capsule to the outside surrounding tumor tissue.

In one embodiment, the biocompatible polymer material disclosed in the present disclosure may be any synthetic or natural polymer with desirable biocompatibility used to replace part of a living system or to function in intimate contact with living tissues/organisms. Biocompatible polymer is intended to interface with biological systems to evaluate, treat, augment, or replace any tissue, organ, or function of a body. The term of “biocompatibility” is used to describe the suitability of a polymer for exposure to the body or body fluids. A polymer is considered biocompatible if it allows the body to function without any complications such as allergic reactions or other adverse side effects. Biocompatible polymer materials are widely used in contact lens, vascular grafts, heart valves, stents, breast implants, renal dialyzers, etc. A biocompatible polymer material may be but not limited to polyethylene glycol (PEG), poly(ethylene oxide) (PEO), poly(alkyl oxazoline) such as poly(ethyl oxazoline) (PEOZ), poly(lactic acid) (PLA), poly(lactic-co-glycolic acid) (PLGA), poly(caprolactone) (PCL), poly(styrene) (PS), poly(alkyl acrylate) such as poly(n-butyl acrylate) (PnBA) or poly(t-butyl acrylate) (PtBA), poly(alkyl methacrylate) such as poly(methyl methacrylate) (PMMA), poly(alkylene carbonate) such as poly(propylene carbonate) (PPC), lipids, or any comonomeric combination thereof. In one aspect, the biocompatible polymer material comprises the reaction product of two or more components that may be but are not limited to polyethylene glycol (PEG), poly(ethylene oxide) (PEO), poly(alkyl oxazoline) such as poly(ethyl oxazoline) (PEOZ), poly(lactic acid) (PLA), poly(lactic-co-glycolic acid) (PLGA), poly(caprolactone) (PCL), poly(styrene) (PS), poly(alkyl acrylate) such as poly(n-butyl acrylate) (PnBA) or poly(t-butyl acrylate) (PtBA), poly(alkyl methacrylate) such as poly(methyl methacrylate) (PMMA), poly(alkylene carbonate) such as poly(propylene carbonate) (PPC), lipids. In one aspect, the biocompatible polymer material may be but not limited to PEG-PLA, PEG-PLGA, PEG-PCL, PEG-PS, PEG-PnBA, PEG-PtBA, PEG-PMMA, PEG-PPC, PEOZ-PLA, PEOZ-PLGA, PEOZ-PCL, PEOZ-PS, PEOZ-PnBA, PEOZ-PtBA, PEOZ-PMMA, PEOZ-PPC, or any combination thereof. In one aspect, the biocompatible polymer material is a block copolymer, which may be but not limited to PEG-PLA, PEG-PLGA, PEG-PCL, PEG-PS, PEG-PnBA, PEG-PtBA, PEG-PMMA, PEG-PPC, PEOZ-PLA, PEOZ-PLGA, PEOZ-PCL, PEOZ-PS, PEOZ-PnBA, PEOZ-PtBA, PEOZ-PMMA, PEOZ-PPC. In one aspect, the biocompatible block copolymer is an amphiphilic block copolymer. In one aspect, the biocompatible block copolymer is an amphiphilic block copolymer that is capable of forming micelles in water, wherein the core domain of the polymer micelle is composed of hydrophobic chains, and the shell layer of the micelle contains hydrophilic chains.

In one embodiment, the biocompatible polymer material disclosed in the present disclosure may be further functionalized with folic acid. In one aspect, the folic acid functionalized biocompatible polymer material may enhance the oral absorption of drugs with poor oral bioavailability, or may have the potential to be used as a carrier for targeted drug delivery in cancer treatment.

In one embodiment, the hydrophobic chemotherapeutic drug disclosed in the present disclosure may be any chemotherapeutic drug that has a water solubility less than about 100 mg/mL, less than 90 mg/mL, less than 80 mg/mL, less than 70 mg/mL, less than 60 mg/mL, less than 50 mg/mL, less than 40 mg/mL, less than 30 mg/mL, less than 20 mg/mL, less than 10 mg/mL, less than 5 mg/mL, or less than 2 mg/mL at room temperature. In one aspect, the hydrophobic chemotherapeutic drug disclosed in the present disclosure may be any chemotherapeutic drug that has a water solubility of 0.0001-100 mg/mL, 0.0001-90 mg/mL, 0.0001-80 mg/mL, 0.0001-70 mg/mL, 0.0001-60 mg/mL, 0.0001-50 mg/mL, 0.0001-40 mg/mL, 0.0001-30 mg/mL, 0.0001-20 mg/mL, 0.0001-10 mg/mL, 0.0001-5 mg/mL, or 0.0001-2 mg/mL at room temperature. Although a chemotherapeutic drug generally refers to a drug for treatment of a cancer, a chemotherapeutic drug in the present disclosure may also refer to a drug used to treat a non-cancer disease such as but not limited to an autoimmune disease or an inflammatory disease. In a different embodiment, two or more different types of hydrophobic chemotherapeutic drugs may be co-encapsulated.

In one embodiment, the hydrophobic chemotherapeutic drug disclosed in the present disclosure may be but not limited to paclitaxel, docetaxel, cabazitaxel, cisplatin, carboplatin, oxaliplatin, nedaplatin, doxorubicin, daunorubicin, epirubicin, idarubicin, gemcitabine, etanidazole, 5-fluorouracil, any salt or derivative thereof, or any combination thereof.

In one embodiment, the radio-luminescent particle or particle aggregate capable of emitting UV, visible, IR light, or a combination thereof under radiation may be but not limited to a metal tungstate material, a metal molybdate material, a metal oxide, a metal sulfide, or a combination thereof. In one aspect, the metal may be but not limited to any suitable alkali metal such as Li, Na, K, Rb or Cs, any suitable alkaline earth metal such as Be, Mg, Ca, Sr, or Ba, any suitable transition metal or poor metal element in the periodic table, or any solvate or hydrate form thereof.

In one embodiment, the radio-luminescent particle or particle aggregate capable of emitting UV, visible, IR light, or a combination under radiation may comprise calcium tungstate (CaWO₄), zinc oxide (ZnO), any solvate or hydrate form thereof, or a combination thereof.

In one embodiment, the radio-luminescent particle or particle aggregate capable of emitting UV, visible, IR light, or a combination thereof under radiation comprises calcium tungstate (CaWO₄).

In one embodiment, the radio-luminescent particle or particle aggregate capable of emitting UV, visible, IR light, or a combination thereof under radiation comprises crystalline radio-luminescent particle or particle aggregate.

In one embodiment, the radio-luminescent particle or particle aggregate is capable of emitting UV under radiation.

In one embodiment, the present disclosure provides a radiation-triggered controlled release drug composition comprising a calcium tungstate (CaWO₄) particle or particle aggregate, paclitaxel, and a biocompatible polymer capsule comprising a block copolymer such as PEG-PLA, PEG-PLGA, PEG-PCL, PEG-PS, PEG-PnBA, or any combination thereof.

In one embodiment, the present disclosure provides that the mean diameter range of said radio-luminescent particle or particle aggregate is between about 1-10,000 nm. In one aspect, the mean diameter range is about 1-1000 nm, 1-900 nm, 1-800 nm, 1-700 nm, 1-600 nm, 1-500 nm, 1-400 nm, 1-300 nm, 1-200 nm, 1-100 nm, 1-90 nm, 1-80 nm, 1-70 nm, 1-60 nm, 1-50 nm, 1-40 nm, 1-30 nm, 1-20 nm, 1-10 nm, or any combination thereof.

In one embodiment, the present disclosure provides that the wavelength range of the UV/visible/IR light generated by the radio-luminescent particle or particle aggregate under radiation may be 10 nm to 100 μm. In one aspect, the wavelength range is 10 nm-10 μm, 10 nm-1 μm, 100 nm-10 μm, 100 nm-1 μm, 100 nm-800 nm, 200 nm-800 nm, 100 nm-700 nm, 200 nm-700 nm, 100 nm-600 nm, 200 nm-600 nm, or any combination thereof.

In one embodiment, the present disclosure provides that the radio-luminescent particle or particle aggregate has a luminescence band gap energy in the range between 1.55 eV (800 nm) and 6.20 eV (200 nm).

In one embodiment, the present disclosure provides that the accumulated amount of released chemotherapeutic drug under radiation is at least 20% greater than the accumulated amount of released chemotherapeutic drug in the absence of radiation over the same period. In one embodiment, the accumulated amount of released chemotherapeutic drug under radiation is at least 30% greater than the accumulated amount of released chemotherapeutic drug in the absence of radiation over the same period. In one embodiment, the accumulated amount of released chemotherapeutic drug under radiation is at least 40% greater than the accumulated amount of released chemotherapeutic drug in the absence of radiation over the same period. In one embodiment, the accumulated amount of released chemotherapeutic drug under radiation is at least 100% greater than the accumulated amount of released chemotherapeutic drug in the absence of radiation over the same period. In one embodiment, the accumulated amount of released chemotherapeutic drug under radiation is at least 200% greater than the accumulated amount of released chemotherapeutic drug in the absence of radiation over the same period. In one embodiment, the accumulated amount of released chemotherapeutic drug under radiation is at least 400% greater than the accumulated amount of released chemotherapeutic drug in the absence of radiation over the same period. In one embodiment, the accumulated amount of released chemotherapeutic drug under radiation is about 40%-400% greater than the accumulated amount of released chemotherapeutic drug in the absence of radiation over the same period. In one embodiment, the accumulated amount of released chemotherapeutic drug under radiation is about 20%-400% greater than the accumulated amount of released chemotherapeutic drug in the absence of radiation over the same period. In one embodiment, the accumulated amount of released chemotherapeutic drug under radiation is about 100%-400% greater than the accumulated amount of released chemotherapeutic drug in the absence of radiation over the same period. In one embodiment, the time period is about 1-40 days, 1-30 days, 1-25 days, 1-20 days, 1-15 days, 1-10 days, 1-5 days, or 1-2 days.

In one embodiment, the present disclosure provides that the radiation comprises ionizing radiation, wherein the ionizing radiation may be but not limited to X-rays, γ rays, electrons, protons, neutrons, ions, or any combination thereof.

In one embodiment, the present disclosure provides that the radiation comprises non-ionizing radiation, wherein the non-ionizing radiation may be but not limited to ultraviolet (UV), visible, or infrared (IR) light, or any combination thereof.

In one embodiment, the present disclosure provides that the radiation comprises ionizing radiation and non-ionizing radiation, wherein the ionizing radiation may be but not limited to X-rays, γ rays, electrons, protons, neutrons, ions, or any combination thereof, wherein the non-ionizing radiation may be but not limited to ultraviolet (UV), visible, or infrared (IR) light, or any combination thereof.

In one embodiment, the present disclosure provides that at least 50% of the chemotherapeutic drug stays within the biocompatible polymer capsule for a period of at least 30 days in the absence of radiation.

It was found that the radio-luminescent particle or particle aggregate may actually suppress the release of the chemotherapeutic drug in the absence of radiation. This was demonstrated by a study that examined the cumulative PTX release properties of non-X-ray-irradiated PTX-encapsulating PEG-PLA micelles with or without co-encapsulated CaWO₄ nanoparticles over 32 days. When the PTX-encapsulating PEG-PLA micelles have no co-encapsulated CaWO₄ nanoparticles, the level of 32-day cumulative PTX release was about 75% of the original amount loaded. When the PTX-encapsulating PEG-PLA micelles have co-encapsulated CaWO₄ nanoparticles, the level of 32-day cumulative PTX release was about 25-30%. Therefore, the radio-luminescent particle or particle aggregate plays an unexpected role in controlling the release kinetics of chemotherapeutic drug from nanoparticles in both X-ray irradiated and non-irradiated situations. More specifically, the radio-luminescent particle or particle aggregate activates a fast release of the chemotherapeutic drug under radiation, whereas it greatly suppresses the release of the chemotherapeutic drug in the absence of radiation. This unexpected radiation-triggered drug release mechanism enables better control of the pharmacokinetics of the chemotherapeutic drug. In one embodiment, the drug release enhancement ratio (DRER, defined as the ratio of the cumulative amount of released PTX in the presence of radiation relative to that in the absence of radiation) is in the range 10-400% over the 1-32 day period. In one embodiment, the DRER is in the range 10-200% over the 1-32 day period. In one embodiment, the DRER is in the range 10-100% over the 1-32 day period. In one embodiment, the DRER is in the range 25-400% over the 1-32 day period. In one embodiment, the DRER is in the range 25-200% over the 1-32 day period. In one embodiment, the DRER is in the range 25-100% over the 1-32 day period. In one embodiment, the DRER is in the range 50-400% over the 1-32 day period. In one aspect, the DRER is in the range 50-200% over the 1-32 day period. In one embodiment, the DRER is in the range 50-100% over the 1-32 day period.

In one embodiment, the present disclosure provides that the release of the chemotherapeutic drug is controlled by the dose and/or frequency of radiation.

In one embodiment, the present disclosure provides a method of treating a disease responsive to the radiation-controlled release drug composition as disclosed in the present disclosure. In one embodiment, the disease is a cancer, wherein the cancer may be but not limited to head and neck cancer, breast cancer, prostate cancer, lung cancer, liver cancer, gynecological cancer, cervical cancer, brain cancer, melanoma, colorectal cancer (including HER2+ and metastatic), bladder cancer, ovarian cancer, and gastrointestinal cancer. Examples of lung cancer include but are not limited to small cell lung cancer (SCLC) and non-small cell lung cancer (NSCLC).

In one embodiment, the present invention provides the use of the radiation-triggered controlled release drug composition as disclosed in the present disclosure in the manufacture of a medicament for the treatment of a cancer as disclosed in the disclosure.

The present disclosure provides pharmaceutical compositions comprising a radiation-triggered controlled release drug composition of the present disclosure, and one or more pharmaceutically acceptable carriers, diluents and/or excipients. Further, the present disclosure provides a method of treating a cancer as disclosed comprising administering to a patient in need thereof a pharmaceutical composition of the present invention.

### Preparation and Characterizations of Drug-Loaded Polymer-Encapsulated Radio-Luminescent Nanoparticles

The general method of preparation of PEG-PLA-encapsulated CWO NPs can be found in WO2016112314A1. This method was adopted to prepare PTX-loaded PEG-PLA-encapsulated CWO NPs (having a mean hydrodynamic diameter of about 50 nm). 300 mg of PEG-PLA block copolymers (BCP) (M_{n,PEG}=5.0 kDa, M_{n,PLA}=5.0 kDa) and 30 mg of PTX were dissolved in 3.8 g of dimethylformamide (DMF, >99.9% purity, Sigma Aldrich) to prepare the first composition. 0.5 mg of CWO NPs (10 nm diameter) was dispersed in 2.1 g of Milli-Q-purified water to prepare the second composition. These two compositions were mixed together rapidly under simultaneous high-speed mechanical stirring (15,000 rpm) and ultrasonication for 30 minutes. The resultant mixture was centrifuged at 4,000 rpm for 10 minutes. The supernatant containing un-encapsulated PTX, excess PEG-PLA and DMF was removed. The precipitate was dried under vacuum oven overnight to produce the PTX-loaded PEG-PLA-encapsulated CWO NPs.

### In Vitro Drug Release Kinetics

To measure the rate of PTX release from PTX-loaded PEG-PLA-coated CWO NPs, the dried pellet obtained from the previous step was re-dispersed in PBS at a CWO concentration of 0.25 mg/ml, and the mixture was placed in a dialysis tube (50 kDa MWCO). The dialysis tube was sealed at both ends, submerged in 50 ml of PBS, and kept under mild stirring using a magnetic stirring bar. PTX release measurements were performed on four samples: (1) X-ray-irradiated PTX-loaded PEG-PLA-encapsulated CWO NPs, (2) non-X-ray-irradiated PTX-loaded PEG-PLA-encapsulated CWO NPs, (3) X-ray-irradiated PTX-loaded PEG-PLA micelles (with no co-encapsulated CWO NPs), and (4) non-X-ray-irradiated PTX-loaded PEG-PLA micelles (with no co-encapsulated CWO NPs). X-ray irradiation was performed at 7 Gy on Day 2 following re-suspension in PBS. At regular intervals, 50 mL of the dialysis medium was taken for measurement of PTX concentration; each time the same volume of blank PBS was added to the medium to compensate for the volume loss. PTX was collected from the dialysis sample by liquid-liquid extraction as described below. 30 mL of dichloromethane (DCM, >99.9% purity, Sigma Aldrich) was added to 100 mL of the dialysis sample. This mixture was vigorously shaken for a few minutes, and then kept undisturbed for 30 minutes until two distinct liquid layers were formed. The bottom DCM solution was carefully collected, and was dried under vacuum oven overnight. The dried substance (PTX) was dispersed in 2 mL of a 1:1 by volume mixture of water and acetonitrile (HPLC solvent), and analyzed by HPLC for determination of the PTX concentration.

### Drug Encapsulation Efficiency

The PTX encapsulation efficiency was defined as: encapsulation efficiency (%)=(amount initially added-amount lost during encapsulation)/(amount initially added)×100. The amount of PTX lost during encapsulation was determined by analyzing the PTX concentration in the supernatant of the centrifuged encapsulation solution by the HPLC method.

### Gel Permeation Chromatography (GPC) Characterization of PEG-PLA Following Exposure to CWO NPs and X-Rays

1.5 mg of PEG-PLA-coated CWO (“PEG-PLA/CWO”) NPs were dispersed in 0.15 mL of PBS. This sample was divided into two portions. One portion was irradiated with a single 7 Gy dose of X-rays (320 keV), while the other portion was not exposed to X-rays. 0.5 mL of dichloromethane (DCM) was added to each of these solutions to extract the PEG-PLA polymer from the aqueous PEG-PLA/CWO suspension. The resulting solutions were vigorously mixed for 10 minutes and centrifuged at 8000 rpm for 10 minutes. The DCM-rich (bottom) phase of the supernatant was collected and dried in a vacuum oven at room temperature for 12 h. The polymer residue was dissolved in HPLC-grade tetrahydrofuran (THF), and the solution was filtered with a 0.22 μm PTFE filter. Both X-ray-treated and non-X-ray-treated polymer samples were analyzed using an AGILENT TECHNOLOGIES® 1200 Series GPC system equipped with a Hewlett-Packard G1362A refractive index (RI) detector and three PLgel 5 μm MIXED-C columns. Tetrahydrofuran (THF) was used as the mobile phase at 35° C. and a flow rate of 1 mL/min. The pristine PEG-PLA was used as control.

### Cell Culture

HN31 cells were provided by MD Anderson Cancer Center. HN31 cells were cultured in Dulbecco's modified eagle's medium supplemented with 10% v/v fetal bovine serum and 0.1% L-glutamine (Gibco Life Technologies) (as recommended by American Type Culture Collection (ATCC)) in a humidified incubator with 5% CO₂ at 37.0° C. Once the cell confluence reached 80%, the growth medium was removed, and adherent cells were washed twice with PBS (Gibco Life Technologies). Cells were then detached from the plates by treatment with 0.05% trypsin/EDTA solution for 4-6 minutes at 37.0° C. Detached cells were centrifuged at 260×g for 7 minutes at room temperature. The cell pellet was resuspended in a minimal amount of growth medium (2-3 ml), and the cells were counted using a haemocytometer. These cells were plated in T-25 cm² flasks (Corning) at a seeding density of 0.2-0.5×10⁶ cells per mL in 5 mL growth medium.

### MTT Cell Viability Assay

The in vitro cytotoxicities of both uncoated CWO NPs (10 nm diameter determined by TEM) and PEG-PLA-coated CWO NPs (50 nm hydrodynamic diameter determined by DLS) in HN31 cells were evaluated using the MTT assay procedure described in the literature. HN31 cells in the exponential growth phase were seeded in a flat-bottom 96-well polystyrene-coated plate at a seeding density of 0.5×10⁴ cells per well, and incubated for 24 hours at 37.0° C. in a 5% CO₂ incubator prior to exposure to CWO NPs. Cells were then treated with various concentrations of PEG-PLA-coated and uncoated CWO NPs (0.16, 0.32, 0.63, 1.25, 2.5 and 5.0 mg CWO per ml solution) (N=5). After 24 hours of incubation, 10 μL of the MTT reagent was added to each well, and further incubated for additional 4 hours. Afterwards, formazan crystals were dissolved by adding 150 μL of a 10% w/v SDS solution to each well, and the absorbances at 570 nm were immediately measured using a microplate reader (BIO-RAD® Microplate Reader-550). The wells with no cells, i.e., containing only the DMEM growth medium, the nanoparticles, and the MTT reagent, were used as the blanks. The wells containing cells (that had not been treated with the nanoparticles) in the medium with the MTT reagent were used as controls.

### Clonogenic Cell Survival Assay

HN31 cells were seeded in 60-mm culture dishes at densities of 0.2×10³ cells per dish for 0 Gy, 1.0×10³ cells per dish for 3 Gy, 2.0×10³ cells per dish for 6 Gy, and 5.0×10³ cells per dish for 9 Gy radiation dose. Samples were prepared in quadruplet for each radiation dose (N=4). Three groups were tested: (1) cells treated for 3 hours with PEG-PLA-coated CWO NPs, (2) cells treated for 3 hours with PTX-loaded PEG-PLA-coated CWO NPs, and (3) untreated cells (control). After 3 hours of nanoparticle treatment, cells were exposed to various doses of 320 keV X-rays at a dose rate of 1.875 Gy per minute (XRAD® 320, Precision X-Ray). Irradiated cells were cultured for 14 days. Colonies resulting from radio-resistant cells were stained with Crystal Violet. Colonies of more than 50 daughter cells in culture were counted (N=4). The Plating Efficiency (PE) and the Survival Fraction (SF) were calculated based on the number of such colonies relative to that of the respective non-irradiated subgroup for each group: PE (%)=(number of colonies survived)/(number of cells initially plated)×100; SF (%)=(PE of a treated group)/(PE of control)×100. Survival fraction(S) vs. radiation dose (D) data were fit to the linear quadratic model, S(D)=exp[−(αD+βD²)], where α and β are fit parameters. The Sensitization Enhancement Ratio (SER) was calculated as the ratio of the X-ray dose needed to obtain 10% survival in untreated cells relative to the dose needed to obtain 10% survival in nanoparticle-treated cells.

### HN31 Tumor Xenografts in NRG Mice

Animal studies were performed in accordance with the guidelines of the American Association for Accreditation of Laboratory Animal Care (AAALAC). Immune-deficient Non-Obese Diabetic (NOD) Rag Gamma (NRG) mice (6-7 weeks old, female) were housed in standard cages within a pathogen-free facility with free access to food and water and an automatic 12-h light-dark cycle. Mice were initially acclimated to the environment for 1 week prior to xenograft implantation. Subcutaneous Head and Neck Squamous Cell Carcinoma (HNSCC) xenografts were produced by implantation 3×106 HN31 cells in 0.1 mL (total volume) of serum-free medium containing 50% MATRIGEL® (BD Bioscience) into the mouse flanks.

### Evaluation of Antitumor Efficacy in Mouse HNSCC Models

Three samples (including the candidate formulations and control) were investigated: (i) PEG-PLA/CWO NPs, (ii) PEG-PLA/CWO/PTX NPs (both in sterile PBS solution), and (iii) blank PBS without NPs (negative control). The efficacy of these formulations was assessed following intratumoral (IT) administration in mouse HN31 xenografts (NRG mice, N=8) both in the presence and absence of X-ray irradiation. HN31 xenografts were prepared as described above. Once the tumor size reached the 100-150 mm³ level, NP formulations (total 100-150 μL solution containing 10 mg/mL of CaWO₄) were IT administered in two portions over two days (at Days 0 and 1) to a final NP concentration of 10 mg CWO per cc tumor. NP-treated tumors were exposed to total 8 Gy fractionated X-Ray doses (with a daily fraction of 2 Gy repeated over 4 consecutive days) (at Days 1-5). The tumor sizes were measured using a digital caliper at regular intervals. The tumor volume was calculated by the formula, V=(π/6)LWH where L, W and H are the length, width and height of the tumor, respectively. Mouse survival analysis was performed using the standard ICH (The International Council for Harmonisation of Technical Requirements for Pharmaceuticals for Human Use) criteria (euthanasia is required if tumor size >2000 cc, or >20% body weight reduction). Following euthanization, tumor tissues were collected and wet weighed. Tumor and organ (liver, spleen, lung, heart, kidney, and brain) specimens were also collected for histology analysis.

### Evaluation of Pharmacokinetics (PK) and Biodistribution (BD) in Mouse HNSCC Models

The PK of the PTX and the BD of CWO NPs were investigated in HN31 xenograft-bearing NRG mice (6 mice per treatment group) following IT administration of PEG-PLA/CWO/PTX NPs; a sample size of 3 mice per group (N=3) was used for the PTX PK evaluation, and the same sample size (N=3) was also used for the CWO BD analysis. The time-dependent PTX concentrations in tumor, blood and other selected tissues were measured by high performance liquid chromatography (HPLC) using a literature procedure, and the time-dependent CWO concentrations in tumor, blood and other selected tissues were measured by atomic absorption spectroscopy (AAS) using a literature procedure. The following specific procedures were used.

Total 42 mice were divided into 7 groups (Groups I-VII) with 6 mice per group. Mice in Groups I-VI received IT injections of PEG-PLA/CWO/PTX NPs, whereas mice in Group VII received only PBS via IT route (control); all procedures were the same as in the efficacy study described above. NP/PBS-injected mice were treated with 2 Gy daily fractions of 320 keV X-rays during first 4 days (i.e., at Days 1, 2, 3 and 4 post NP injection, up to total 8 Gy X-ray dose). Groups I, II, III, IV, V and VI was sacrificed by euthanization at Day 1, 3, 5, 7, 14 and 30, respectively. The cumulative X-Ray doses mice received were 2 Gy for Group I, 4 Gy for Group II, and 8 Gy for all other Groups (III-VI). Control mice (Group VII) were euthanized at Day 1. Blood samples were collected before euthanization. Tumor and organ (liver, spleen, kidney, lungs, brain, and heart) were collected after euthanization. Tissue samples were processed using literature procedures for HPLC and AAS analyses.

### Statistical Analysis

All in vitro measurements were performed in minimum triplicates. Different animal numbers were chosen for different in vivo assays based on our experience and needs in terms of statistical significance. All data are presented as mean±standard deviation. A one-way ANOVA was used to determine whether there was a statistically significant difference in effect between different treatment groups. Kaplan-Meier survival analysis was used to plot unadjusted survival of mice treated with different formulations; results were analyzed using the log-rank test. Difference was considered statistically significant if p<0.05 (*) and highly significant if p<0.01 (**).

### Results

### Determination of PTX Concentration by HPLC

An HPLC procedure was developed to quantitate PTX released from nanoparticles. A C18 column with dimensions 4×125 mm (AGILENT® 1100 HYPERSIL®, 5 μM) was used as the stationary phase. A 60:40 by volume mixture of water and acetonitrile was used as the mobile phase at a flow rate 1.0 mL/min. The sample injection volume was 10 μL. The PTX absorbance was measured using a UV detector at 204 nm wavelength. Standard solutions containing different concentrations of PTX in the range of 10-1000 μg/mL were prepared from a concentrated stock solution. PTX concentrations were estimated using an isocratic reverse phase HPLC method. From these data, a calibration plot was prepared relating UV absorbance to PTX concentration. The linear relationship could be represented by y=8.9569·x (R²=0.9998), wherein y represents the UV adsorption at 204 nm (mAu), and x represents the PTX concentration (μg/mL).

### Paclitaxel Release Kinetics

The amount of PTX released from PEG-PLA-coated CWO NPs was measured by HPLC for 32 days; both X-ray-irradiated and non-irradiated samples were tested. As control, PTX released from PEG-PLA micelles (containing no co-encapsulated CWO NPs) was also quantitated. It was found that in the absence of radiation, PEG-PLA/CWO/PTX NPs showed the lowest PTX release; about 71% PTX remained unreleased at Day 32. In contrast, upon exposure to 7 Gy X-Ray dose, a sudden burst release of PTX was observed (that is, >50% of the initially loaded PTX amount was released within 2 days following X-ray irradiation, and only about 10% PTX remained unleased at Day 32); this radiation-triggered burst release phase was followed by a slower release phase over the remaining non-irradiated period. In contrast, in the PTX-loaded PEG-PLA micelle case (involving no co-encapsulated CWO NPs), the PTX release profile was significantly less affected by X-ray irradiation (in the absence of radiation about 26% PTX remained unreleased at Day 32, and X-ray treatment slightly decreased this number to about 19%). It should be noted that the presence of CWO NPs significantly suppressed PTX release. This result suggests that PTX may have strong affinity to CaWO₄. On the other hand, this attractive interaction between PTX and CaWO₄ appears to become ineffective under X-ray irradiation. In the process of radiation-triggered PTX release from PEG-PLA/CWO/PTX NPs, UV-A light generated by CWO NPs under X-ray irradiation may play a certain important role in causing a burst release of PTX. X-ray irradiation itself may also directly trigger the release of PTX. A detailed study suggests that indeed both types of radiation can contribute to the release of the drug (further discussed in a later section below).

### Multi-Compartmental Model for Predicting In Vivo Pharmacokinetics (PK) of Intratumorally Injected PTX

Intratumoral chemo-radio combination therapy involves two steps: (1) intratumoral injection of PTX-loaded PEG-PLA-encapsulated CWO NPs, and (2) X-ray irradiation of the nanoparticle-treated tumor. The dynamics of intratumoral PTX concentration can be modeled with reasonable fidelity using a simplistic multi-compartmental PK model. Key kinetic processes involved can be summarized as follows. Radiation directly or indirectly triggers the release of PTX from the polymer coating layer inside the tumor; in the absence of radiation, the PTX release is very slow. Released PTX will accumulate in the tumor compartment. On the other hand, there is continuous loss of PTX to the tumor exterior (e.g., by diffusion). The PTX eliminated from the tumor mainly enters the cardiovascular circulatory system, and eventually becomes cleared from the body through the kidneys.

In clinics, patients with locally advanced head and neck squamous cell carcinomas (HNSCC) typically undergo radiotherapy at a total radiation dose of 66-74 Gy. The protocol is that the total dose is distributed over a period of 40-50 days in 2 Gy daily fractions (5 fractions per week on week days with rest on weekends). PTX PK simulations were performed under this exact same radiation dose setting. It was assumed that the solid tumor had a volume of 100 cc (assumed to be invariant over time), and the tumor was initially injected with three different doses of PEG-PLA/CWO/PTX NPs (2, 5 or 10 mg CWO per mL of tumor). The initial PTX concentration in the PLA coating layer was fixed at 20% by weight for all calculations. The X-ray dose used was 70 Gy, divided into 2 Gy daily fractions (with 5 fractions per week and rest on weekends as in clinical practice). The intraparticle, intratumoral and intracirculatory PTX PK profiles were traced for 210 days (≈7 months); all radiation sessions were completed by Day 47, and no radiation was given in the remaining period. Previously, the tumor elimination rate constant for PTX intratumorally delivered to mouse xenografts in the polymer encapsulated form has been reported: ket=0,005 h⁻¹. A slightly lower tumor PTX elimination constant value (k_{e,t}≈0.001 h⁻¹) was assumed for PEG-PLA/CWO/PTX NPs and PEG-PLA/PTX micelles considering that spontaneous (human) tumors have a denser tissue structure. The intratumoral PTX concentration was calculated as a function of time by solving the following differential kinetic equation, ([rate of PTX accumulation within tumor]=[rate of PTX release from nanoparticles]−[rate of PTX elimination from tumor (e.g., due to diffusion to surrounding tissue, metabolization, etc.)]):

$$\frac{dC}{dt} = k ( {C}_{s} - C ) - {k}_{e , t} C \qquad ( 1 )$$

In the above equation, C is the PTX concentration within the tumor (in Molar units), Cₛ is the PTX concentration within the PLA “shell” layer (in Molar units), k is the rate constant for PTX release from the PLA layer (h⁻¹), and k_{e,t} is the rate constant for PTX elimination from the tumor (h⁻¹). The initial condition used was: C=0 at t=0. Cₛ is coupled to C by the mass balance:

$${C}_{s} {V}_{s} = {C}_{s , o} {V}_{s , o} - {\int }_{0}^{t} k ( {C}_{s} - C ) Vdt \qquad ( 2 )$$

In the above equation, Vₛ is the total volume of the PLA layers within the tumor, V is the volume of tumor, C_{s,o}=Cₛ(t=0), and V_{s,o}=Vₛ(t=0); for simplicity, it was assumed that V and Vₛ did not change with time (i.e., V=100 cc, and Vₛ=V_{s,o} at all times). Therefore, Equation (1) was actually solved simultaneously together with Equation (2) to obtain predictions for C and Cₛ as functions of time.

These computations were carried out for three different types of PTX formulation (PEG-PLA/CWO/PTX, PEG-PLA/PTX, and TAXOL®) under various initial nanoparticle/PTX dose conditions (0.2, 0.5 and 1.0 mg PTX per cc tumor). Note the in vitro IC₉₀ value of PTX (i.e., PTX concentration giving rise to 90% cell kill in vitro) has been reported to be about 90 μg/mL.

The results of this study showed that in the presence of CaWO₄, radiation triggered PTX release, and the intratumoral PTX concentration showed an increasing trend during the initial phase of treatment involving radiation (i.e., for the first 47 days). This initial boost in PTX dose helped in prolonging the PTX availability within the tumor above the therapeutic threshold (e.g., IC90) throughout and beyond the radiotherapy session. The tumor availability of intratumorally administered PTX was significantly influenced by the total initial amount of PTX injected. However, at an identical total amount of PTX injected, it was obvious that the PEG-PLA/CWO/PTX system was able to maintain the therapeutic PTX level for a much longer period of time (e.g., by >25 days at 1 mg/cc PTX dose) than the PEG-PLA/PTX system; in the PEG-¬-PLA/CWO/PTX case the intratumoral PTX level was maintained above the IC90 for about 130 days, whereas in the PEG-PLA/PTX case the intratumoral PTX level was maintained above the IC90 only for about 103 days (in the TAXOL® case the intratumoral PTX level fell below the IC90 within much less than a day).

It was also found that the PTX-loaded PEG-PLA micelle system exhibited an initial burst release; the drug release rate was very high initially (between Days 0 and 10), dropped rapidly afterward, and became stagnant for the rest of the period; the PEG-PLA/PTX system released about half of the loaded PTX within the first 10 days. Although “burst release” has positive aspects (immediate therapeutic effects, easier to overcome drug resistance, etc.), it is generally considered a downside because it is difficult to avoid even when such effect is not desired. To the contrary, in the presence of co-encapsulated CWO NPs (i.e., in the PEG-PLA/CWO/PTX system), the initial burst PTX release phase was not observed. Instead, radiation could be used to create a short period of rapid (burst) PTX release on demand in a highly controlled manner (e.g., >50% PTX released within a couple of days following 7 Gy radiation). In the PEG-PLA/CWO/PTX case, PTX release can be externally controlled by radiation; radiation dose and frequency influence PTX release.

Therefore, this radiation-controlled PTX release mechanism may enable to maintain PTX tumor levels in the therapeutic range for a longer period (e.g., for >120 days at 1 mg/mL PTX dose). PTX intratumorally delivered in the form of TAXOL® remained in the tumor, for instance, for <12 hours at a PTX dose of 10 mg/mL.

It was also found that the PTX concentration in the PLA layer of a PEG-PLA/CWO/PTX or PEG-PLA/PTX nanoparticle decreased with time. It was observed that in the PEG-PLA/PTX case, the PTX concentration in the PLA layer dropped rapidly in the initial “burst release” phase (0-10 days), followed by a second phase of much slower PTX release. In the PEG-PLA/CWO/PTX case, radiation enabled to extend the period of rapid release to about 50 days; about 70% of initially loaded PTX was released from the PLA layer during this rapid release (i.e., radiotherapy) period. Consequently, the tumor PTX concentration was maintained at therapeutic levels for a longer period of time.

It is reasonable to expect that after leaving the tumor, PTX will be mainly absorbed by the (blood) circulatory system. It is useful to estimate the PTX concentration in the circulatory system; high levels of PTX in the blood could produce systemic toxicity. The PTX concentration in the blood could be calculated using the mass balance equation:

$$\frac{d ( {C}_{b} {V}_{b} )}{dt} = {k}_{e . t} CV - {k}_{e . b} {C}_{b} {V}_{b} \qquad ( 3 )$$

In Equation (3), Cb is the PTX concentration in the blood, Vb is the total blood volume in humans (≈4700 mL in a healthy adult human male, and ke.b is the rate constant for PTX renal clearance in humans (≈0.336±0.002 h-1). The results of simulations for three different types of PTX formulation (PEG-PLA/CWO/PTX, PEG-PLA/PTX, and TAXOL®) under various initial nanoparticle/PTX dose conditions (0.2, 0.5 and 1.0 mg PTX per cc tumor) were obtained. At an identical initial PTX dose, the PTX concentration in the blood for the PEG-PLA/CWO/PTX system was higher than that for the PEG-PLA/PTX system. A typical PTX dose in systemic chemotherapy is about 200 mg/m2 in humans, which translates into a value of about 100 in the units of μg PTX per mL blood (based on the blood volume of 4700 mL for a healthy adult human male). This PTX dose level causes dermatological side effects (in skin, hair, nail, etc.) in 86.8% of the patients treated, and cognitive/mental health-related problems in 75% of patients treated. The blood concentration of PTX intratumorally administered using the PEGPLA/CWO/PTX (or PEG-PLA/PTX) delivery system was several orders of magnitude below this toxic threshold, which, therefore, supports that the intratumoral chemo-radio therapy proposed in this document will not, indeed, produce systemic chemo drug side effects. The blood concentration of PTX delivered in the form of TAXOL® peaked at a few minutes post-administration (for instance, at a level of about 0.4 μg/mL within about 6 minutes following IT administration at an initial PTX dose of 10 mg per cc of tumor), and was significantly higher than PTX delivered using the PEG-PLA/CWO/PTX or PEG-PLA/PTX formulation.

### Photo-Lytic Degradation of PLA

As depicted in FIG. 1, in the radiation-triggered controlled release drug formulation the radio-luminescent CWO NPs are coated with PEG-PLA block copolymers. Hydrophobic PLA chains form a globular domain wherein CWO NPs are encapsulated. Hydrophilic PEG chains form a hydrated brush layer. Water-insoluble PTX molecules are co-encapsulated within the hydrophobic PLA domain. Under X-ray irradiation, UV-A is generated by CWO NPs, and for some reason, this process causes the release of PTX from the PLA coating layer into the aqueous surrounding. The PTX release triggered by X-rays may be due to the degradation of the PLA polymer that occurs under X-ray irradiation. In order to confirm the degradation of PLA under X-ray irradiation, GPC measurement was performed on the PEG-PLA re-extracted with chloroform from PEG-PLA-coated CWO NPs following exposure to X-rays (320 keV, 7 Gy) (“PEG-PLA/CWO+X-Ray”). For comparison, the same measurements were also performed on pristine PEG-PLA (“PEG-PLA”) and the PEG-PLA re-extracted from non-X-ray-exposed PEG-PLA-coated CWO NPs (“PEG-PLA/CWO”). It was found that no difference in GPC curves was observed between “PEG-PLA” and “PEG-PLA/CWO”. However, the X-ray-exposed sample (“PEG-PLA/CWO+X-ray”) showed a large broadening of the peak on the longer elution time (lower molecular weight) side, which clearly indicates that the degradation of the polymer occurred; the PTX release triggered by X-ray radiation was thus due to the chemical degradation of the encapsulating polymer (not due to physical excitation processes).

To better understand the exact mechanism of the PLA degradation in X-ray-irradiated PEG-PLA/CWO NPs, another set of GPC measurements were made on (i) pristine PEG-PLA (“PEG-PLA”) (a repeat experiment using a replicate PEG-PLA material), (ii) the PEG-PLA re-extracted from PEG-PLA-coated CWO NPs following exposure to X-rays (320 keV, 7 Gy) (“PEG-PLA/CWO+X-Ray”) (a repeat experiment using replicate PEG-PLA and CWO NP materials), (iii) the PEG-PLA re-extracted from PEG-PLA-coated CWO NPs following exposure to UV-A light (365 nm, 0.561 J/cm², equivalent 365 nm UV-A fluence generated by PEG-PLA/CWO NPs under 7 Gy 320 keV X-ray radiation) (“PEG-PLA/CWO+UV-A”), (iv) the PEG-PLA re-extracted from empty (non-CWO-loaded) PEG-PLA micelles following exposure to X-rays (320 keV, 7 Gy) (“PEG-PLA+X-Ray”), and (v) the PEG-PLA re-extracted from empty (non-CWO-loaded) PEG-PLA micelles following exposure to UV-A light (365 nm, 0.561 J/cm²) (“PEG-PLA+UV-A”). The results showed that both X-rays alone (“PEG-PLA+X-Ray”) and UV-A light alone (“PEG-PLA+UV-A”) caused PLA degradation even in the absence of CWO NPs. Further, the extents of PLA degradation were comparable between “PEG-PLA+UV-A” and “PEG-PLA/CWO+UV-A”, and also between “PEG-PLA+X-Ray” and “PEG-PLA/CWO+X-Ray”. These results indicate that CWO does not produce any significant catalytic activity for PLA degradation (likely because of insufficient availability of oxygen or water molecules within the PLA domain); the PLA degradation is therefore not of photo-catalytic type, but it is a photo-lysis reaction.

### Low-Cytotoxicity of PEG-PLA CWO NPs

In vitro cytotoxicities of uncoated CWO NPs (10 nm diameter) and PEG-PLA-encapsulated CWO NPs (50 nm mean hydrodynamic diameter) were evaluated in HN31 (p53-mutant human head and neck cancer) cells using the standard MTT protocol (N=3) at various CWO concentrations ranging from 0.16 to 5 mg/ml. No significant toxicity was observed for both samples up to 1.25 mg/mL. At higher concentrations (2.5 and 5 mg/ml), a slight reduction (10-20%) in viability was observed. It should be noted that the actual CWO concentration that the cells experience is typically significantly higher than the nominal value of CWO concentration because of the sedimentation of the CWO NPs. These results support that CWO NPs, regardless of whether PEG-PLA-coated or uncoated, have low cytotoxicity, and therefore may be safe for clinical use.

Clonogenic Survival Following Various Doses of Radiation in HN31 Cells Treated with Concurrent PEG-PLA/CWO/PTX NPs

An in vitro clonogenic study was performed to determine whether PEG-PLA/CWO/PTX NPs are capable of inducing a significant enhancement of the tumor suppressive effect of X-rays/y rays beyond what is achievable with PEG-PLA/CWO NPs (i.e., without co-delivered PTX). Again, the HN31 cell line was used for this investigation.

HN31 cells were irradiated in the presence of PEG-PLA/CWO/PTX NPs or PEG-PLA/CWO NPs (CWO concentration: 0.20 mg/ml). HN31 cells were seeded on 60 mm culture plates at densities 0.2×10³ (0 Gy), 1.0×10³ (3 Gy), 2.0×10³ (6 Gy) and 5×10³ (9 Gy) cells per plate. After 24 h incubation with nanoparticles, cells were exposed to various doses of 320 keV X-ray radiation. Irradiated cells were cultured for 14 days. Colonies resulting from radio-resistant cells were stained by Crystal Violet. Colonies of more than 50 daughter cells in culture were counted (N=4). Table 1 summarizes the linear quadratic fit results and the SER (Sensitization Enhancement Ratio estimated at 10% clonogenic survival) values.

TABLE 1

| | α | β | α/β | SER |
|---|---|---|---|---|
| Control | 0.159 | 0.044 | 3.61 | 1 |
| PEG-PLA/CWO NP | 0.266 | 0.039 | 6.82 | 1.15 |
| PEG-PLA-PTX/CWO NP | 0.436 | 0.036 | 12.11 | 1.40 |

The parameters α and β are the linear-quadratic exponential fit parameters. The Sensitization Enhancement Ratio or SER is defined as the ratio of the radiation dose at 10% clonogenic survival in the absence of CWO relative to the radiation dose at 10% survival in the presence of CWO.

The clonogenic survival curves for radiated HN31 cells (regardless of whether X-rays were used alone or in combination with concomitant PEG-PLA/CWO/PTX or PEG-PLA/CWO NPs) were seen to follow the standard exponential-quadratic decay formula S(D)=exp[−(αD+βD²)]. In the formula, S is the survival fraction, D is the X-ray dose, and α and β are adjustable parameters for fitting data to the model. The results are summarized in Table 1. Also, the α/β ratio has a useful meaning; this ratio represents a radiation dose at which the exponential-linear cell kill effect becomes equivalent in magnitude to the exponential-quadratic cell kill effect of radiation (at D<α/β the exponential-linear effect is dominant, whereas at D>α/β the exponential-quadratic effect takes over (the surviving fraction drops more rapidly)).

It is generally known that cells that respond to radiation early have high α/β ratios. Cell kill linearly increases at low radiation doses. The average value of α/β for early responding cells is about 10. Cells that respond late have low α/β ratios. Cell kill is less at low doses, and greatly increases at high doses. The average value of α/β for late responding cells is about 3. Most tumor cells have high α/β ratios (equal to or greater than 10). However, some tumor types exhibit much lower ratios; for instance, prostate and melanoma/sarcoma typically show α/β values around 3 and 1, respectively. Tumors with low α/β ratios are resistant to low doses of radiation.

As shown in Table 1, concomitant PEG-PLA/CWO/PTX NPs significantly increased the value of the α/β ratio (α/β=12.11) relative to non-nanoparticle-treated control (α/β=3.61), which suggests that the PEG-PLA/CWO/PTX treatment enhanced the radio responsiveness of HN31 cells at low X-ray doses. Therefore, it may be deduced that PTX released from nanoparticles under X-ray irradiation contributed to overall cell kill by increasing radiotherapy efficacy (i.e., by radio sensitization) in addition to functioning as chemotherapy. It should also be noted that, though lesser in degree than PEG-PLA/CWO/PTX, non-PTX-loaded PEG-PLA/CWO NPs also increased both SER and α/β, which supports that PEG-PLA/CWO NPs themselves are also an effective radio-sensitizer.

### Therapeutic Efficacy of PEG-PLA/CWO/PTX NPs in Mouse HN31 Xenografts: Tumor Growth, and Mouse Survival

The therapeutic efficacy of intratumorally administered PEG-PLA/CWO/PTX NPs was evaluated in HN31 mouse xenografts in vivo. For these studies, mice were treated via intratumoral injection with either PTX-loaded (“PEG-PLA/CWO/PTX”) NPs, non-PTX-loaded (“PEG-PLA/CWO”) NPs or NP vehicle (PBS). Each treatment/control group was divided into two subgroups; one subgroup was treated with X-rays (320 keV, total 8 Gy, 4 fractions of 2 Gy given one fraction per day), and the other was not given X-rays. Tumor growth and mouse survival were measured over time.

Tumor growth in mice treated with concomitant radiation plus PEG-PLA/CWO/PTX NPs was measured. NRG mice (6-8 weeks old, female, N=8) were implanted with HN31 cells at Day 0. Tumors were grown to 0.10 to 0.15 cc until Day 5. Nanoparticles were intratumorally administered in 2 portions at Days 5 and 6 post HN31 implantation. Tumors were irradiated with 320 keV X-rays (total dose 8 Gy) in 2 Gy/day fractions over 4 days (at Days 6, 7, 8 and 9 post HN31 implantation). Control group was treated with sterile PBS. For all treatment types (PEG-PLA/CWO/PTX, PEG-PLA/CWO, and Control), non-X-ray-treated animals were also included in the study for comparison.

It was found that 8 Gy radiation caused a significant decrease in tumor growth; for instance, tumor growth was significantly suppressed in the “PBS+X-Ray” group relative to the “PBS” group. Most importantly, a concomitant treatment with PEG-PLA/CWO/PTX NPs produced a significant enhancement of the tumor suppressive effect of X-rays; for instance, at 17 days post HN31 implantation, the tumor volumes (mean±standard deviation, N=8) were measured to be 665±108 mm³ for “PBS”, 664±47 mm³ for “PEG-PLA/CWO”, 711±142 mm³ for “PEG-PLA/CWO/PTX”, 251±28 mm³ for “PBS+X-Ray”, 241±37 mm³ for “PEG-PLA/CWO+X-Ray”, and 137±21 mm³ for “PEG-PLA/CWO/PTX+X-Ray”.

Kaplan-Meier curves were constructed for survival of mice (N=8) treated with PEG-PLA/CWO/PTX NPs, PEG-PLA/CWO NPs, and PBS (control) with and without X-rays. PBS solutions of NPs were injected into HN31 xenografts (0.10-0.15 cc) in NRG mice to a final NP concentration of 10 mg of CWO per cc of tumor. A total radiation dose of 8 Gy was given in 4 fractions of 2 Gy per fraction, one fraction per day over 4 days (at t=1, 2, 3 and 4 days) following NP administration (at t=0 and 1 day). Mice were euthanized based on the standard ICH criteria: (a) tumor volume >2.0 cc; (b) body weight loss >20% of the original body weight. Analysis of survival data was performed using the log-rank test. Values of p<0.05 were considered statistically significant. The PEG-PLA/CWO/PTX+X-Ray group and PEG-PLA/CWO+X-Ray group were significantly different from the Control (PBS with no X-Ray) group and also from the NPs with no X-Ray groups (p<0.05 for each pair-wise comparison). The median survival times were: 18 days for “PBS”, 22 days for “PEG-PLA/CWO”, 22 days for “PEG-PLA/CWO/PTX”, 28 days for “PBS+X-Ray”, 37 days for “PEG-PLA/CWO+X-Ray”, and 45 days for “PEG-PLA/CWO/PTX+X-Ray”.

It is notable that PEG-PLA/CWO/PTX NPs plus X-rays increased the mouse survival by about 8 days relative to the “PEG-PLA/CWO+X-Ray” treatment. Log-rank analysis confirmed that the survival benefit produced by “PEG-PLA/CWO/PTX+X-Ray” is statistically significant relative to any other treatment: “PEG-PLA/CWO+X-Ray” (p=0.00008), “PBS+X-Ray” (p=0.0007), “PEG-PLA/CWO/PTX” (p=0.0001), “PEG-PLA/CWO” (p=0.00005), and “PBS” (p=0.00002). Overall, these results clearly support the therapeutic potential of the concurrent X-ray and “PEG-PLA/CWO/PTX” therapy.

### Biodistribution of PEG-PLA/CWO/PTX NPs in Tumor-Bearing Mice Following Intratumoral Administration

A biodistribution (BD) study was performed to evaluate whether PEG-PLA/CWO/PTX NPs stay localized at the solid tumor site for the duration of a normal course of radiation therapy (25-40 days) following intratumoral administration in the HN31 xenograft mouse model. A long tumor residence time of PEG-PLA/CWO/PTX NPs (>one month) will enable a single injection of these nanopartciels at the beginning of treatment period to replace multiple daily/weekly injections of standard chemo radio-sensitizers. Complete retention of NPs within the infused tumor region is also key to controlling the PTX availability within the tumor and minimizing systemic side effects. In this study, 42 mice were divided into 7 groups of 6 mice each (6 treatment groups, and one control group). All mice in treatment groups received an identical treatment, i.e., an intratumoral injection of PEG-PLA/CWO/PTX NPs (to a final NP concentration of 10 mg CWO per cc tumor, injected in 2 portions at t=−1 and 0 days) following by X-ray radiation (320 keV, 4 fractions of 2 Gy per day over 4 days, i.e., at Days 0, 1, 2 and 3); the treatment details were the same as in the efficacy study discussed above. The control group was treated with vehicle (PBS) only (with no radiation therapy) and sacrificed at Day 1. Animals in different groups were euthanized at different time points (t): t=1 day (Group I, exposed to 2 Gy radiation on Day 0), t=3 days (Group II, exposed to 2+2 Gy radiation on Days 0 and 1, respectively), t=5 days (Group III, exposed to 2+2+2 Gy radiation on Days 0, 1 and 2, respectively), t=7 days (Group IV, exposed to 2+2+2+2 Gy radiation on Days 0, 1, 2 and 3, respectively), t=15 days (Group V, exposed to 2+2+2+2 Gy radiation on Days 0, 1, 2 and 3, respectively), and t=30 days (Group VI, exposed to 2+2+2+2 Gy radiation on Days 0, 1, 2 and 3, respectively)). Tumor, blood and organ (brain, heart, kidney, lung, liver and spleen) samples were collected, and analyzed for calcium (Ca) content by atomic absorption spectroscopy (AAS) (N=3). The rest 3 mice from each group were used to evaluate the pharmacokinetics of PTX released from X-ray-irradiated PEG-PLA/CWO/PTX NPs, as will be discussed in the next Section. The results confirmed that the CWO NPs remained localized in the tumor for (at least) 30 days after injection. Over this one-month measurement period, intratumoral CWO NP retention was maintained at a virtually constant level around about 80% with statistical fluctuations (approximately ±15%) due to measurement uncertainties (N=3). Also, of note, negligible amounts of CWO NPs were detected in other organs within uncertainties associated with small sample sizes (N=3).

Pharmacokinetics of PTX Released from Intratumorally Injected PEG-PLA/CWO/PTX NPs in Tumor-Bearing Mice Following X-Ray Irradiation

In the study described in the previous section, half the animals from each group (N=3) were also used to determine the pharmacokinetic (PK) distribution of PTX in the tumor, blood and major organs (brain, heart, kidney, lung, liver, and spleen) by HPLC. The results showed that approximately 70% of the injected PTX amount still remained in the tumor for 7 days, about 50% for 15 days, and about 25% for one month; note that the measured intratumoral PTX amount represents the sum of the amount of the drug released from the polymer but retained within the tumor plus the amount remaining (unreleased) in the polymer matrices. Although the absolute amount of PTX dropped only by a factor a little over 3 times (from 86% at Day 1 to 25% at Day 30), the decrease in the intratumoral concentration of PTX was far more pronounced (from 63 μg/mg at Day 1 to 5 μg/mg at Day 30), because of the rapid increase in tumor size). Most notably, even at one month post injection, the intratumoral PTX concentration (5 μg/mg) was still two orders of magnitude greater than the in vitro IC₉₀ value of PTX(≈0.09 μg PTX per mg tumor. Further, the PK behavior of the PTX can be quantitatively described by the multi-compartmental PK model with no adjustable parameters (i.e., solely on the basis of experimental rate constants), which supports the validity of the predictions of the model for human tumors. The level of the PTX in blood and other organs was below the HPLC detection limit at all times examined. Taken together, in vivo results quantitatively validate the favorable pharmacological properties of PEG-PLA/CWO/PTX NPs (therapeutic efficacy, high intratumoral drug availability, low systemic drug levels).

The present disclosure demonstrates radiation-controlled drug release nanoparticle formulations (“PEG-PLA/CWO/PTX NPs”) as a means to achieve maximum bioavailability and minimum adverse effects of the chemo drugs (PTX), and also their ability to affect head and neck cancer cells (in vitro) and xenografts (in vivo).

This radiation-controlled drug release method will enable patients with advanced solid tumors to achieve the benefits of chemo-radio combination treatment with reduced negative effects. This approach also presents a new therapeutic option that has not previously been available for pateints excluded from conventional chemo-radiotherapy protocols. Those skilled in the art will recognize that numerous modifications can be made to the specific implementations described above. The implementations should not be limited to the particular limitations described. Other implementations may be possible.

We claim:

1. A method comprising:
a) providing a composition directly into tumor tissue, the composition comprising one or more CaWO₄ (CWO) nanoparticles co-encapsulated with one or more chemotherapeutic drugs in the same capsule, the capsule comprising PEG-PLA block-copolymers (BCPs); and
b) providing a dose of X-ray radiation to the tumor comprising the composition, the X-ray radiation causing:
(i) degradation of the capsule when the one or more CWO nanoparticles emit at least UV-A light; and
(ii) release of the chemotherapeutic drugs from the inside of the capsule to the tumor tissue.

2. The method of claim 1, wherein the chemotherapeutic drug is an anti-cancer agent having a water solubility less than about 100 mg/mL at room temperature.

3. The method of claim 1, wherein the chemotherapeutic drug is paclitaxel, docetaxel, cabazitaxel, cisplatin, carboplatin, oxaliplatin, nedaplatin, doxorubicin, daunorubicin, epirubicin, idarubicin, gemcitabine, etanidazole, 5-fluorouracil, any salt or derivative thereof, or any combination thereof.

4. The method of claim 1, wherein the chemotherapeutic drug is paclitaxel.

5. The method of claim 1, wherein the mean diameter of the composition is in the range between about 1 nm and about 10,000 nm.

6. The method of claim 1, further comprising one or more pharmaceutically acceptable carriers, diluents and/or excipients.
